(19) United States
(12) Patent Application Publication
(10) Pub. No.: US 2011/0040196 A1
(43) Pub. Date: Feb. 17, 2011

(54) BLOOD PRESSURE MONITOR

(75) Inventors: Yi-Cheng Shih, Chiayi City (TW); Feng-Chi Liu, Taipei County (TW); Yi-Chin Tsai, Taipei County (TW); Shih-Chieh Yen, Taipei County (TW); Kwan-Chiao Liao, Taipei County (TW); Shih-Yan Hung, Taipei City (TW)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: QUANTA COMPUTER INC., Tao Yuan Shien (TW)

(21) Appl. No.: 12/619,894

(22) Filed: Nov. 17, 2009

(30) Foreign Application Priority Data
Aug. 12, 2009 (TW) 98214831

(51) Int. Cl. A61B 5/022

(52) U.S. Cl. 600/493

(57) ABSTRACT

A blood pressure monitor for measuring a blood pressure of a human body includes a main body, a connecting mechanism, and a sensing unit. The main body surrounds the human body and has a first end portion and a second end portion. The connecting mechanism connects the first end portion with the second end portion at a first position or a second position. When the first end portion and the second end portion are connected at the first position, the human body surrounded thereby has a first circumference, and when the first end portion and the second end portion are connected at the second position, the human body surrounded thereby has a second circumference. The sensing unit, disposed on the body, detects the first position or the second position to determine the first circumference or the second circumference respectively.

This Application claims priority of Taiwan Patent Application No. 098214831 filed on Aug. 12, 2009, the entirety of which is incorporated by reference herein.

### BACKGROUND OF THE INVENTION

1. Field of the Invention

The invention relates to a blood pressure monitor, and in particular, to a blood pressure monitor which is able to measure the circumference of a human body.

2. Description of the Related Art

When measuring blood pressure, many factors affect the measuring result. In addition to the characteristics ratio, the circumference of a measured human body is also an important factor which needs to be taken into consideration when measuring blood pressure. However, conventional electronic blood pressure monitors, such as upper arm blood pressure monitors, wrist blood pressure monitors, and a finger blood pressure monitor, do not consider the characteristics ratio and the circumference of the measured human body in blood pressure measurement results, thereby resulting in inaccurate measurements.

### BRIEF SUMMARY OF THE INVENTION

A blood pressure monitor for measuring a blood pressure of a human body is provided. The blood pressure monitor includes a main body, a connecting mechanism, and a sensing unit. The main body surrounds the human body and has a first end portion and a second end portion. The connecting mechanism connects the first end portion with the second end portion at a first position or a second position. When the first end portion and the second end portion are connected at the first position, the human body surrounded thereby has a first circumference, and when the first end portion and the second end portion are connected at the second position, the human body surrounded thereby has a second circumference. The sensing unit, disposed on the body, detects the first position or the second position to determine the first circumference or the second circumference, respectively.

A detailed description is given in the following embodiments with reference to the accompanying drawings.

### BRIEF DESCRIPTION OF THE DRAWINGS

The invention can be more fully understood by reading the subsequent detailed description and examples with references made to the accompanying drawings, wherein:

FIG. 1A is a schematic view of a blood pressure monitor of a first embodiment of the invention;

FIG. 1B is a schematic view showing another perspective of the blood pressure monitor in FIG. 1A;

FIG. 2 is a schematic view showing another perspective of the blood pressure monitor in FIG. 1A;

FIG. 3 is a schematic view of the blood pressure monitor in FIG. 2, showing the connection between a first end portion and a second end portion;

FIG. 4A is a schematic view of a blood pressure monitor of a second embodiment of the invention;

FIG. 4B is an exploded view of the blood pressure monitor in FIG. 4A;

FIG. 5 is a schematic view of an operating member of the second embodiment of the invention; and

FIGS. 6A and 6B are schematic views showing the operation of the blood pressure monitor of the second embodiment of the invention.

### DETAILED DESCRIPTION OF THE INVENTION

### First Embodiment

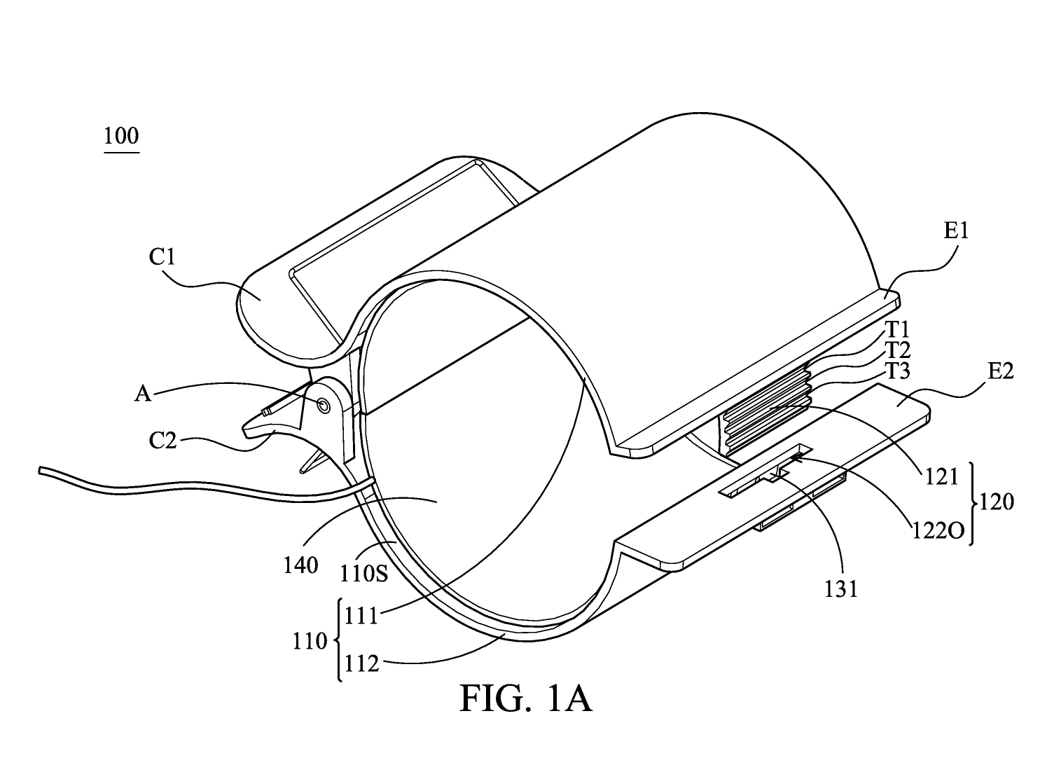

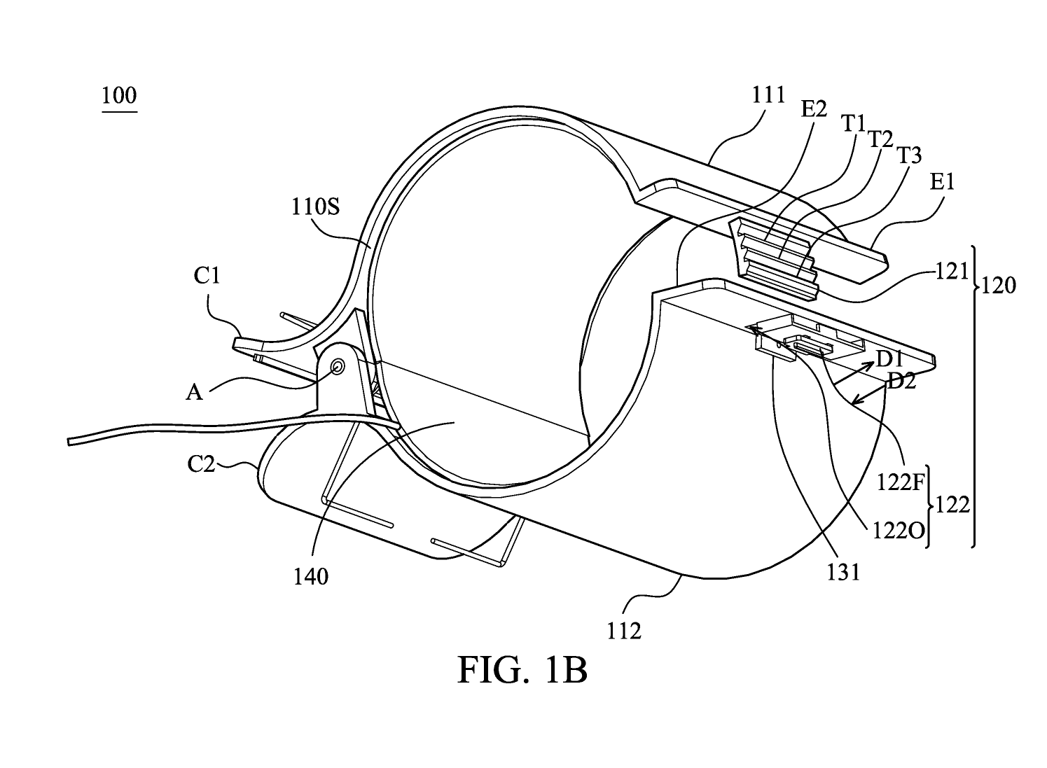

Referring to FIGS. 1A and 1B, in a first embodiment of the invention, the blood pressure monitor 100, an upper arm blood pressure monitor, comprises a main body 110, a connecting mechanism 120, a sensing member 131, an inflation bag 140, a controller (not shown) and a display unit (not shown). The user places his arm into the main body 110 to measure blood pressure from the upper arm. The blood pressure monitor 100 calculates the blood pressure value according to the measured data and the parameter of the circumference of the upper arm by the controller, and displays the blood pressure value on the display unit.

The main body 110 comprises a first body 111 and a second body 112. The first body 111 comprises a first end portion E1 and a first connecting end portion C1 opposite to the first end portion E1. The second body 112 comprises a second end portion E2 and a second connecting end portion C2 opposite to the second end portion E2. The first connecting end portion C1 and the second connecting end portion C2 are pivotally connected according to an axle A, such that the first body 111 and the second body 112 are able to pivot relatively to each other, and the first end portion E1 relatively moves close to or away from the second end portion E2 accordingly.

The connecting mechanism 120 comprises a first connecting portion 121 and a second connecting portion 122. The first connecting portion 121, disposed on the first end portion E1, comprises a first engaging portion T1, a second engaging portion T2 and a third engaging portion T3. The second connecting portion 122, disposed on the second end portion E2, has an opening 122O and a corresponding engaging portion 122F (as shown in FIG. 1B). The opening 122O is formed on the second end portion E2, and the corresponding engaging portion 122F is movably disposed beneath the opening 122O. After the first connecting portion 121 penetrates through the opening 122O, the corresponding engaging portion 122F moves forth along a first direction D1 and back along the second direction D2 to selectively engage with or be separated from one of the first engaging portion T1, the second engaging portion T2 or the third engaging portion T3, wherein the first direction D1 is opposite to the second direction D2 (as shown in FIG. 1B).

In detail, the first end portion E1 and the second end portion E2 are connected at a first position, which is defined when the corresponding engaging portion 122F engages with the first engaging portion T1, the first end portion E1 and the second end portion E2 are connected at a second position, which is defined when the corresponding engaging portion 122F engages with the second engaging portion T2, and the first end portion E1 and the second end portion E2 are connected at a third position, which is defined when the corresponding engaging portion 122F engages with the third engaging portion T3,

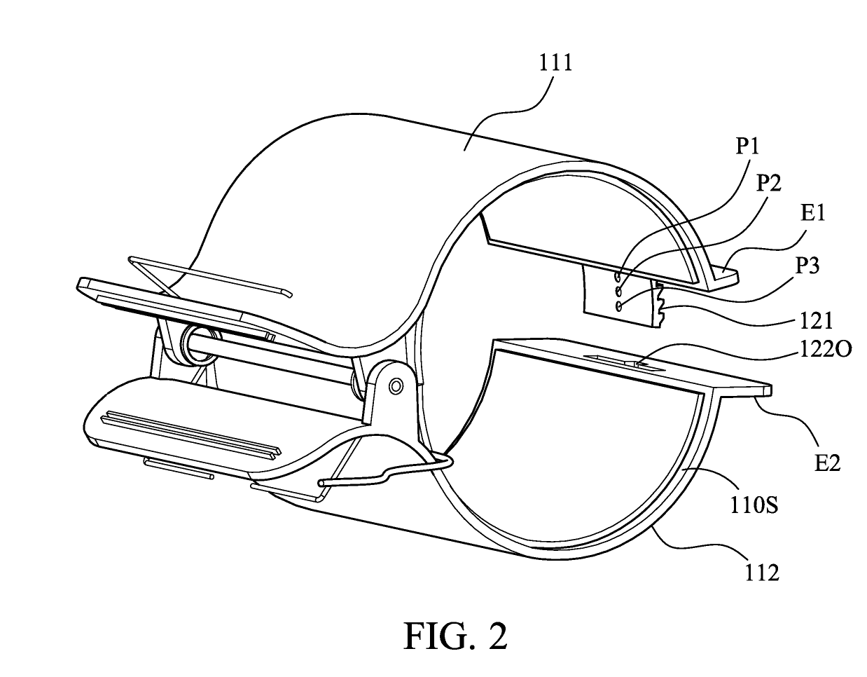

Referring to FIG. 1B and FIG. 2, the sensing member 131 electrically connects with the controller, and is disposed on the second end portion E2. Three sensing points P1, P2, P3 are located on the inner side of the first connecting portion 121 (i.e. opposite to the side where the engaging portions are disposed on), and respectively corresponds to the sensing member 131 when the first end portion E1 and the second end portion E2 are connected at the first position, the second position and the third position. Thus, the sensing member 131 obtains different data according to the different sensing points (P1, P2, and P3) and transmits the different data to the controller for calculating the blood pressure.

It should be noted that the first connecting portion 121 may comprise a fourth engaging portion or more according to requirement, such that the first end portion E1 and the second end portion E2 may be connected at various position. The number of the sensing points also has to be added according to the number of engaging portions.

Additionally, the main body 100 has a curved inner surface 1105, and the inflation bag 140 is disposed along the inner surface 1105 of the main body 110. When the upper arm is placed in the main body 110, the inflation bag 140 is inflated to surround tightly the upper arm to help measuring of blood pressure.

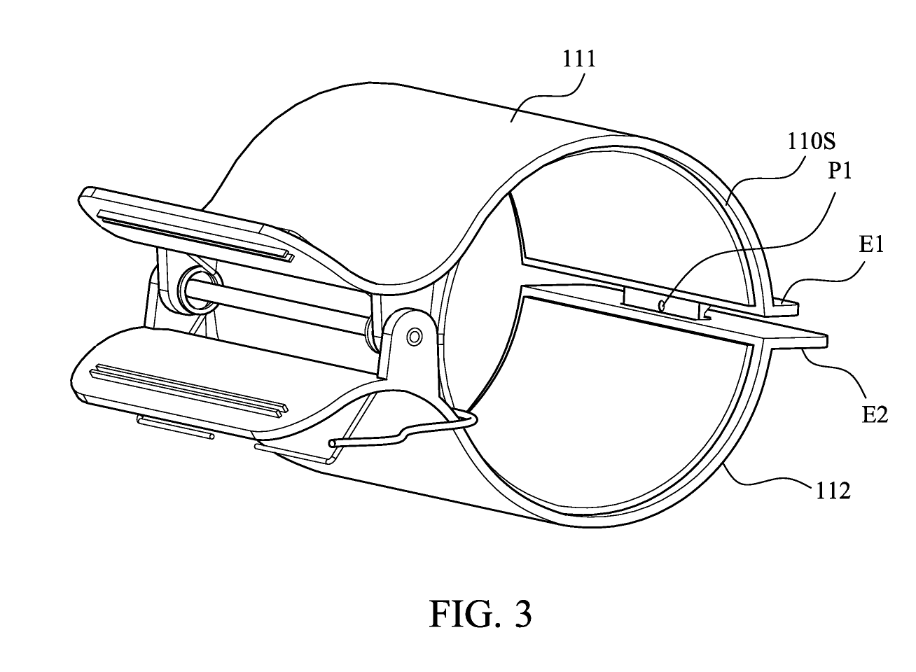

Referring to FIG. 1B and FIG. 3, before the measuring of blood pressure, the user first moves the corresponding engaging portion 122F along the first direction D1, and then moves the first connecting portion 121 toward the second connecting portion 122 to adjust the connection between the first end portion E1 and the second end portion E2 according to the circumference of the upper arm. Specifically, after the first connecting portion 121 penetrates through the opening 122O, the corresponding engaging portion 122F moves along the second direction D2 to engage with one of the first engaging portion T1, the second engaging portion T2 or the third engaging portion T3 of the first connecting portion 121. Thus, the sensing member 131 is corresponded to one of the sensing points (P1, P2 or P3). For example, when the sensing member 131 is corresponding to the first sensing point P1, the sensing member 131 reads the information regarding to the sensing point P1 and transmits the information to the controller. According to pre-stored data saved in the controller, a first circumference corresponding the information regarding to the sensing point P1 is determined. In other words, the circumference of the user's upper arm approximately equals to the first circumference. For the same reason, when the sensing member 131 corresponds to the second sensing point P2 or the third sensing point P3, the sensing member 131 reads the information regarding to the second sensing point P2 or the third sensing point P3 and transmits the information to the controller to determine a second circumference or a third circumference of a user's arm. After the circumference of the upper arm is known, the controller may precisely calculate blood pressure.

### Second Embodiment

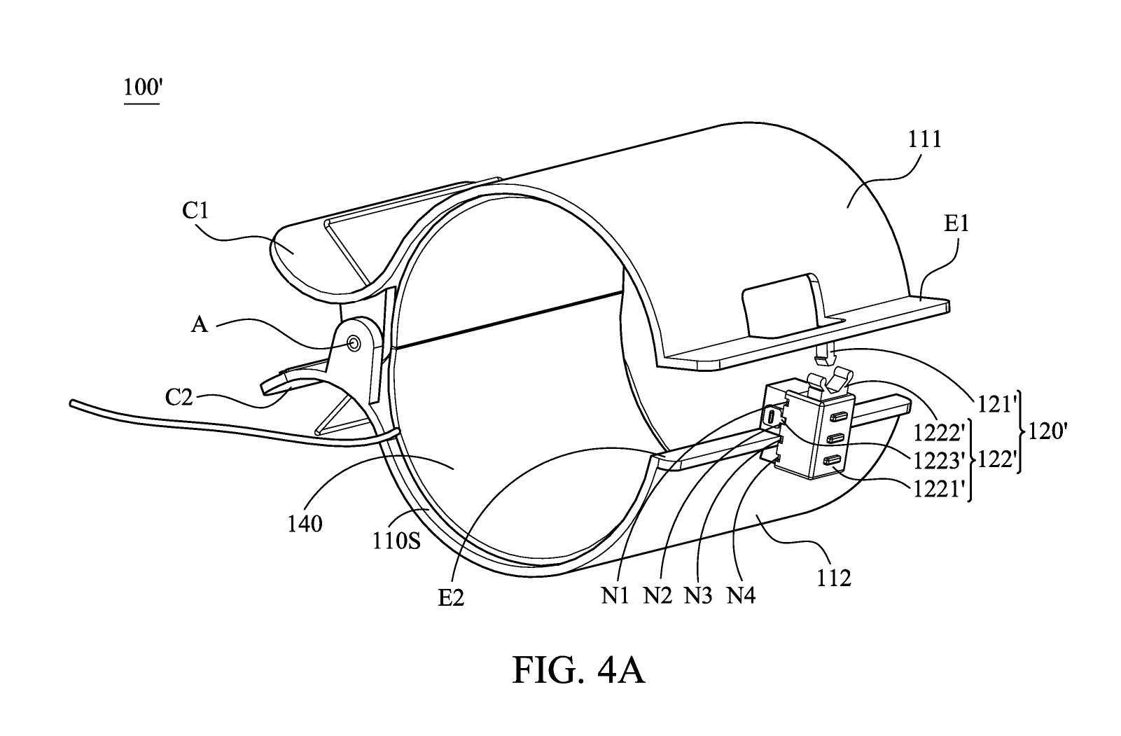

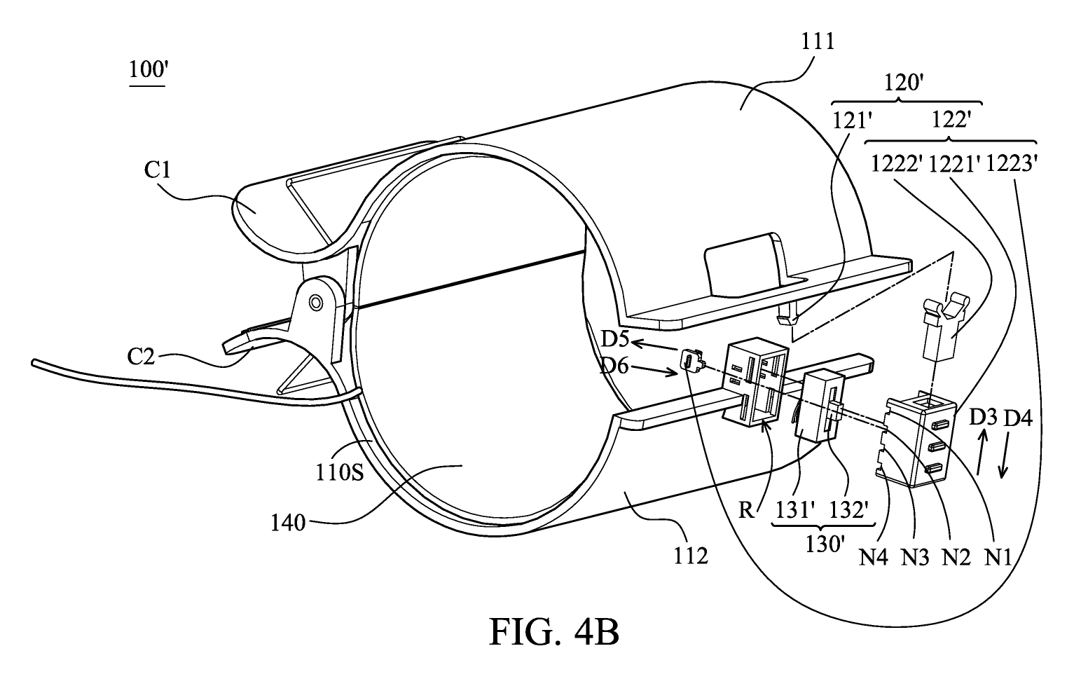

Referring to FIGS. 4A and 4B, in the second embodiment of the invention, the blood pressure monitor 100′ comprises the same components as those in the first embodiment except for the connecting mechanism 120′ and the sensing unit 130′. Therefore, the description of the same components is omitted in the following description.

Referring to FIGS. 4A and 4B, as shown in FIG. 4A, the connecting mechanism 120′ comprises a first connecting portion 121′ and a second connecting portion 122′. The second connecting portion 122′ comprises an operating member 1221′, a holding member 1222′ and a fixing member 1223′. As shown in FIG. 4B, the sensing unit 130′ comprises a sensing body 131′ and a moving member 132′.

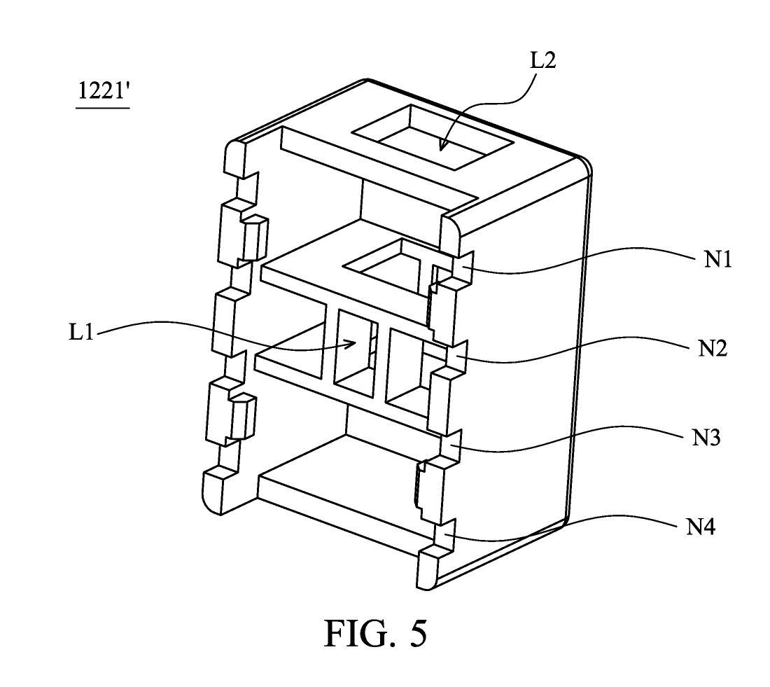

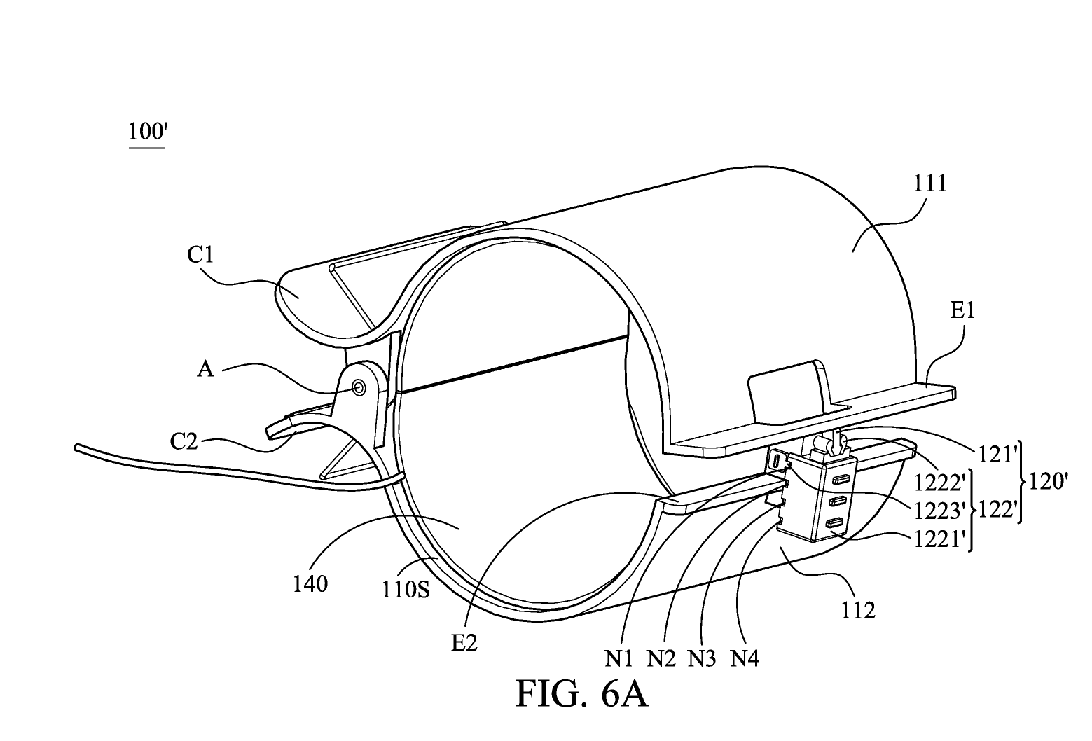

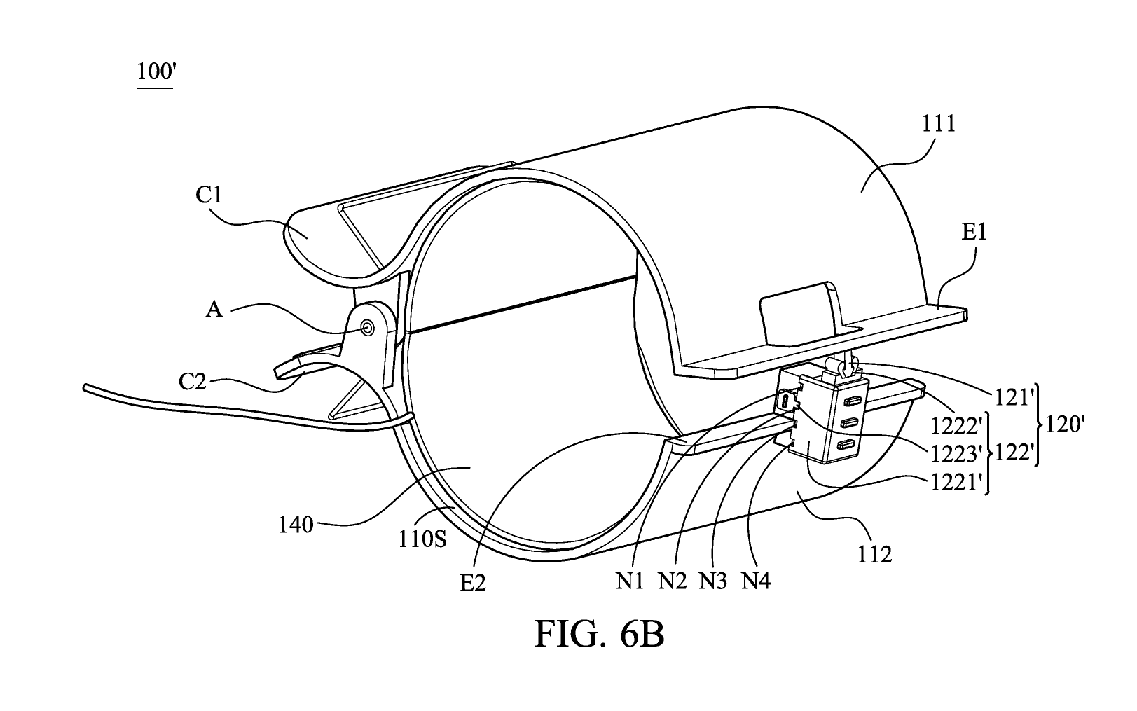

Referring to FIGS. 4A, 4B and FIG. 5, the first connecting portion 121′ is fixed on the first end portion E1. The operating member 1221′, movably disposed on the second end portion E2, moves along a third direction D3 or a fourth direction D4 (the third direction D3 and the fourth direction D4 are opposite directions) and has a first notch L1 and a second notch L2. Note that a first recess N1, a second recess N2, a third recess N3 and a fourth recess N4 are formed on the edge of the operating member 1221′. The holding member 1222′, fixed in the second notch L2 of the operating member 1221′, is used to hold the first connecting portion 121′ (as shown in FIGS. 6A and 6B). When the operating member 1221′ moves along the third direction D3 or the fourth direction D4, the holding member 1222′ and the first connecting portion 121′ are drived to move along the third direction D3 and the fourth direction D4, thus the first end portion E1 is able to move close to or away from the second end portion E2 at a first position, a second position, a third position or a fourth position due to the connection between the first connecting portion 121′ and the operating member 1221′. The fixing member 1223′, disposed on a side of the receiving portion R on the second end portion E2, moves along a fifth direction D5 or a sixth direction D6 (the fifth direction D5 and the sixth direction D6 are opposite directions) to engage with the operating member 1221′. That is, when the operating member 1221′ moves along the third direction D3 or the fourth direction D4 to connect the first end portion E1 with the second end portion E2 at different positions, the fixing member 1223′ corresponds to the different recesses (N1, N2, N3, and N4) accordingly, and engages with the corresponding recesses to fix the position of the operating member 1221′. In detail, when the fixing member 1223′ engages with the first recess N1, the first end portion E1 and the second end portion E2 are connected at the first position, when the fixing member 1223′ engages with the second recess N2, the first end portion E1 and the second end portion E2 are connected at the second position, when the fixing member 1223′ engages with the third recess N3, the first end portion E1 and the second end portion E2 are connected at the third position, and when the fixing member 1223′ engages with the fourth recess N4, the first end portion E1 and the second end portion E2 are connected at the fourth position.

The receiving portion R (as shown in FIG. 4B) on the second end portion E2 is for receiving the sensing body 131′ of the sensing unit 130′. The moving member 132′ is movably disposed on the sensing body 131′ and embedded in the first notch L1 of the operating member 1221′. Therefore, when the operating member 1221′ moves along the third direction D3 or the fourth direction D4, the moving member 132′ is moved relatively to the sensing body 131′. The sensing body 131′ detects the position of the moving member 132′ and sends the position information to the controller.

As shown in FIG. 6A, the fixing member 1223′ engages with the first recess N1 of the operating member 1221′ to fix the connection between the first end portion E1 and the second end portion E2 at the first position. As shown in FIG. 6B, the fixing member 1223′ engages with the second recess N2 of the operating member 1221′ to fix the connection between the first end portion E1 and the second end portion E2 at the second position. The operating member 1221′ further comprises the third recess N3 and the fourth recess N4. When the fixing member 1223′ engages with the third recess N3 or the fourth recess N4 of the operating member 1221′, the first end portion E1 and the second end portion E2 can be fixedly connected at the third or fourth position.

It should be noted that in the embodiment, the first end portion E1 and the second end portion E2 are connected at four different positions, but it is not limited thereto. The operating member 1221′ may comprise more moving positions according to requirement, and the number of the recesses on the operating member 1221′ may be adjusted according to moving positions of the operating member 1221′.

Before measuring of blood pressure, the user first moves the fixing member 1223′ along the fifth direction D5, and then moves the operating member 1221′ along the third direction D3 or the fourth direction D4 to adjust the connection between the first end portion E1 and the second end portion E2 to a proper position (the first position, the second position, the third position or the fourth position) according to the circumference of the upper arm. Finally, the fixing member 1223′ is moved along the sixth direction D6 to fix the operating member 1221′. During the movement of the operating member 1221′, the moving member 132′ of the sensing unit 130′ are moved along with the operating member 1221′, and the sensing body 131′ detects the stop (or final) position of the moving member 132′, which is also the stop position of the operating member 1221′. For example, when the first end portion E1 and the second end portion E2 are connected at the first position, the sensing body 131′ reads the information of the stop position of the moving member 132′ and transmits the information to the controller. According to pre-stored data saved in the controller, a first circumference corresponding to the stop position of the moving member 132′ is determined. In other words, the circumference of the user's upper arm approximately equals to the first circumference. For the same reason, when the first end portion E1 and the second end portion E2 are connected at the second position, the third position or the fourth position, a second, third or fourth circumference corresponding to the stop position of the moving member 132′ is determined.

It should be noted that in the embodiment, the blood pressure monitor 100, 100′ is an upper arm blood pressure monitor which measures blood pressure and the circumference from the upper arm, but it is not limited thereto. The blood pressure monitor 100, 100′ of the invention can also be a wrist blood pressure monitor or a finger blood pressure monitor which measures the circumference of the human body.

The blood pressure monitor of the invention measures the circumference of the upper arm, and provide the circumference to the controller for fine tuning, thus an accurate blood pressure could be obtained an displayed on the display unit electrically connected to the controller optionally.

While the invention has been described by way of example and in terms of preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A blood pressure monitor, measuring a blood pressure of a human body, comprising:
a main body, for surrounding the human body, having a first end portion and a second end portion;
a connecting mechanism, for connecting the first end portion with the second end portion at a first position or a second position at a first position or at a second position, when the first end portion and the second end portion are connected at the first position, the human body surrounded thereby has a first circumference, and when the first end portion and the second end portion are connected at the second position, the human body surrounded thereby has a second circumference; and
a sensing unit, disposed on the body and detecting the first position or the second position to determine the first circumference or the second circumference.

2. The blood pressure monitor as claimed in claim 1, wherein the main body comprises:
a first body having a first connecting end portion; and
a second body having a second connecting end portion pivotally connected to the first connecting end portion.

3. The blood pressure monitor as claimed in claim 2, wherein the first end portion is opposite to the first connecting end portion, and the second end portion is opposite to the second connecting end portion.

4. The blood pressure monitor as claimed in claim 1, wherein the connecting mechanism comprises:
a first connecting portion disposed on the first end portion and having a first engaging portion and a second engaging portion; and
a second connecting portion disposed on the second end portion and having a corresponding connecting portion, the corresponding connecting portion selectively engages with the first engaging portion or the second engaging portion;
wherein when the corresponding engaging portion engages with the first engaging portion, the first end portion and the second end portion are connected at the first position, and when the corresponding engaging portion engages with the second engaging portion, the first end portion and the second end portion are connecting at the second position.

5. The blood pressure monitor as claimed in claim 4, wherein the first engaging portion and the second engaging portion are notches, and the corresponding engaging portion is movably disposed on the second end portion.

6. The blood pressure monitor as claimed in claim 4, wherein the second connecting portion further comprises an opening, and after the first connecting portion penetrates the opening, the corresponding engaging portion engages with the first engaging portion or the second engaging portion.

7. The blood pressure monitor as claimed in claim 5, wherein the corresponding engaging portion is disposed at the edge of the opening.

8. The blood pressure monitor as claimed in claim 4, wherein the first connecting portion further comprises:
a first sensing point, corresponding to the sensing unit when the first end portion and the second end portion being connected at the first position; and
a second sensing point, corresponding to the sensing unit when the first end portion and the second end portion being connected at the second position.

9. The blood pressure monitor as claimed in claim 1, wherein the blood pressure monitor further comprises:
a first sensing point located on the first end portion, and corresponding to the sensing unit when the first end portion and the second end portion being connected at the first position; and
a second sensing point located on the first end portion, and corresponding to the sensing unit when the first end portion and the second end portion being connected at the second position.

10. The blood pressure monitor as claimed in claim 1, wherein the connecting mechanism comprises:
a first connecting portion fixed on the first end portion; and
a second connecting portion movably disposed on the second end portion and connecting with the first end portion;
wherein the second connecting portion moves to adjust the connection between the first end portion and the second end portion at the first position or the second position.

11. The blood pressure monitor as claimed in claim 10, wherein the sensing unit comprises:
a sensing body disposed on the second end potion; and
a moving member movably disposed on the sensing body, engaging with the second connecting portion, and being movable with the second connecting portion,
wherein when the second connecting portion moves to adjust the connection between the first end portion and the second end portion at the first position or the second position, the sensing body senses a position of the moving member to determine the first circumference or the second circumference.

12. The blood pressure monitor as claimed in claim 11, wherein the second connecting portion comprises:
an operating member movably connecting with the second end portion, such that the first end portion and the second end portion are adjusted thereby to be connected at the first position or the second position; and
a holding member disposed in the operating member for holding the first connecting portion;
wherein when the operating member moves, the holding member, the first connecting portion and the moving member are moved accordingly.

13. The blood pressure monitor as claimed in claim 12, wherein the second connecting portion further comprises a fixing member movably disposed on the second end portion, and selectively engages with or separates from the operating member.

14. The blood pressure monitor as claimed in claim 13, wherein the operating member has a plurality of recesses, and the fixing member engages with one of the recesses.

15. The blood pressure monitor as claimed in claim 12, wherein the operating member comprises a first notch, and the first notch engages with the moving member.

16. The blood pressure monitor as claimed in claim 1, wherein the operating member comprises a second notch, and the second notch receives the holding member.

17. The blood pressure monitor as claimed in claim 1, further comprising a micro control unit electrically connected to the sensing unit.

18. The blood pressure monitor as claimed in claim 1, wherein the second end portion has a receiving portion to receive the sensing unit.

19. The blood pressure monitor as claimed in claim 1, wherein the main body has a curved inner surface.

20. The blood pressure monitor as claimed in claim 19, further comprising a inflation bag, disposed on the inner surface of the main body.
